SEQUENCE LISTING (9 sequences)

SEQ ID NO: 1 - protein, 679 aa, Homo sapiens
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 2 - protein, 692 aa, Homo sapiens
GRRRRSVQWCTVSQPEATKCFQWQRNMRRVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNDAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 3 - protein, 679 aa, Artificial Sequence
  Y188F mutant
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGFSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 4 - protein, 679 aa, Artificial Sequence
  Y95F/Y188F mutant
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFFYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGFSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 5 - protein, 679 aa, Artificial Sequence
  Y426F/Y517F mutant
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGFFAVAVVKKSASDLTWDNLKGKKSCHTAVGRTAGWNIPMGLLYNKINHCRFDEFFSEGCAPGSKKDSSLCKLCMGSGLNLCEPNNKEGYYGFTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 6 - protein, 255 aa, Homo sapiens
MFHQVRRVMTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 7 - protein, 202 aa, Homo sapiens
MQSLPNSNGAAAGRDFKMKLWDVVAVCLVLLHTASAFPLPAANMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 8 - protein, 200 aa, Homo sapiens
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 9 - protein, 38 aa, Homo sapiens
HSDGIFTDSYSRYRKQMAVKKYLAAVLGKRYKQRVKNK

(19) United States
(12) Patent Application Publication
(10) Pub. No.: US 2023/0263863 A1
(43) Pub. Date: Aug. 24, 2023

(54) COMPOSITIONS HAVING NEUROREGENERATIVE APPLICATIONS

(71) Applicant: GRIFOLS WORLDWIDE OPERATIONS LIMITED, DUBLIN (IE)

(72) Inventors: Thomas BARNETT, Research Triangle Park, NC (US); David A. ROSS, Research Triangle Park, NC (US)

(21) Appl. No.: 18/004,231

(22) PCT Filed: Jul. 7, 2021
(86) PCT No.: PCT/EP2021/068873

§ 371 Date: Jan. 4, 2023

Related U.S. Application Data
(60) Provisional application No. 63/049,486, filed on Jul. 8, 2020.

(51) Int. Cl. A61K 38/40; A61P 25/28; A61P 25/16

(52) U.S. Cl. CPC A61K 38/40; A61P 25/28; A61P 25/16

(57) ABSTRACT

Pharmaceutical compositions containing transferrin or lactoferrin for use in promoting or inducing the generation new neural cells in a patient that has suffered a neurodegenerative event. The neurodegenerative event may be caused by a neurodegenerative disease such as Alzheimer's, Parkinson's, Huntington's, or amyotrophic lateral sclerosis. Ideally, the transferrin and/or lactoferrin have a low iron saturation.

### FIELD OF THE INVENTION

The present invention relates to therapeutic proteins and their use in the field of regenerative medicine. In particular, disclosed herein are applications of transferrin and lactoferrin and their use in promoting the proliferation, induction, and/or differentiation of neural progenitor cells or neural stem cells.

### BACKGROUND

Neurodegenerative diseases, such as amyotrophic lateral sclerosis, Huntington's disease, Alzheimer's disease, and Parkinson's disease, are illnesses characterised by progressive neuronal cell death and are associated with high morbidity, patient distress, low quality of life, and high mortality rates. As population demographics shift increasingly towards the older end of the spectrum the prevalence of neurodegenerative conditions within society is rapidly climbing. With life expectancy continually rising it is envisaged that neurodegenerative conditions will compete with cancer and cardiovascular diseases as a leading cause of death for future generations.

Without exception, at the time of writing, there are no approved treatments that can cure or reverse the effects of neurodegenerative diseases. In certain circumstances there are approved drugs that delay the progression of the disease. For example, riluzole (RILUTEK) and edaravone (RADICAVA) are two approved therapies for the treatment of amyotrophic lateral sclerosis that delay the progression of the disease, however the molecules do not reverse the symptoms of the disease once they have manifested.

Given the lack of curative therapies it is not surprising that most commercially approved treatments are symptomatic in focus, such as inter alia dopaminergic treatments for Parkinson's disease and movement disorders, antipsychotic drugs for behavioural and psychological symptoms of dementia, and analgesic drugs for the management of pain.

Neuroprotection is an alternative, non-restorative approach to the management of neurodegenerative diseases. The scientific and patent literature abounds with reports of neuroprotective compounds and molecules that attempt to limit the damage caused by neurodegenerative diseases and slow their debilitating progress.

One such example is U.S. Patent Publication No. 2016008437 in the name of Grifols Worldwide Operations Ltd which discloses mixtures of apo-transferrin and holo-transferrin exerting a neuroprotective effect by modulating the activity of Hypoxia Inducible Factors (HIF) in a number of degenerative disease states. Similarly, International Patent Application Publication No. WO2006/20727 to HealthPartners Research Foundation proposes the use of deferoxamine as a modulator of Hypoxia Inducible Factor-1α to elicit a neuroprotective response against the harmful effects of reperfusion in ischemic patients.

Notwithstanding the foregoing it is immediately apparent that there is a paucity of clinical candidates having the potential to cure or reverse the debilitating effects of neurodegenerative diseases and conditions. Approved clinical therapies are confined to managing symptoms of the diseases and there remains an unmet need for treatments that do more than slow the progression of the conditions via neuroprotective effects and pathways. Innovations having the ability to cure or at least partially reverse neural damage remain elusive and, as such, are highly desirable.

### DESCRIPTION OF THE INVENTION

The words “comprises/comprising” and the words “having/including” when used herein with reference to the present invention are used to specify the presence of stated features, integers, steps or components but do not preclude the presence or addition of one or more other features, integers, steps, components, or groups thereof.

It should be appreciated by those skilled in the art that the specific embodiments disclosed herein should not be read in isolation, and that the present specification intends for the disclosed embodiments to be read in combination with one another as opposed to individually. As such, each embodiment may serve as a basis for modifying or limiting other embodiments disclosed herein.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “10 to 100” should be interpreted to include not only the explicitly recited values of 10 to 100, but also include individual value and sub-ranges within the indicated range. Thus, included in this numerical range are individual values such as 10, 11, 12, 13 . . . 97, 98, 99, 100 and sub-ranges such as from 10 to 40, from 25 to 40 and 50 to 60, etc. This same principle applies to ranges reciting only one numerical value, such as “at least 10”. Furthermore, such an interpretation should apply regardless of the breadth of the range or the characteristics being described.

Methods of Treatment

In a first aspect, the present invention provides for a method of promoting and or inducing the generation of new neural cells in a patient that has suffered a neurodegenerative event, the method comprising administering a therapeutically effective amount of a protein selected from transferrin, lactoferrin, and combinations thereof to the patient in need thereof.

The skilled person will appreciate that among the plethora of mammalian iron-binding proteins transferrin and lactoferrin are related proteins of the transferrin family with 61% sequence identity. In addition to a number of overlapping and complimentary functions, transferrin and lactoferrin also demonstrate a number of mutually exclusive functions. The present invention includes within its scope all wild type mammalian transferrin proteins, however, human transferrin (UniProtKB Seq. No. Q06AH7) comprising the amino acid sequence set forth in SEQ ID NO: 1 is particularly preferred. Similarly, the present invention includes within its scope all wild type mammalian lactoferrin proteins, however, human lactoferrin (UniProtKB Seq. No. P02788) comprising the amino acid sequence set forth in SEQ ID NO: 2 is particularly preferred.

The wild type transferrin protein contains two homologous lobes (N- and C-lobes) with each lobe binding a single iron atom. As such, each wild type transferrin molecule can bind up to two iron atoms or ions per molecule. Similarly, each wild type lactoferrin molecule can bind two iron atoms per molecule in an analogous fashion.

Transferrin and lactoferrin can be extracted from natural sources or alternatively manufactured using a recombinant production/manufacturing process. Suitable natural sources may be human plasma or human milk respectively.

By “transferrin” the current specification is to be construed as meaning a therapeutically effective amount of:
a wild type (mammalian, preferably human) transferrin protein,
a functional mutant thereof,
a functional fragment thereof, or
combinations thereof.

The iron saturation of the transferrin, functional mutant thereof, or functional fragment thereof may be about 50% or less. Preferably, the iron saturation is about 40% or less. In one embodiment, the iron saturation is about 30% or less. For example, the iron saturation may be about 20% or less, such as about 10% or less. In some embodiments, the iron saturation is about 5% or less. In yet a further embodiment, the iron saturation may be less than about 1%. For the avoidance of any doubt, ranges presented herein as less than X % include 0 to X %, i.e. transferrin with absolutely no bound iron—0% iron saturation.

As used herein, “apo-transferrin” shall mean transferrin having an iron saturation of less than 1%. Similarly, “holo-transferrin” shall mean transferrin having an iron saturation of 99% or greater.

The skilled person will appreciate that transferrin iron saturation levels can be readily determined without undue burden by quantifying the total iron levels in a sample having a known transferrin concentration. The total iron levels in sample can be measured by any one of a number of methods known by those of skill in the art.

Suitable examples include:
Colorimetric assays—Iron is quantitated by measuring the intensity of the violet complex formed in the reaction between ferrozine and Fe²⁺ in acetate buffer at 562 nm. Thiourea or other chemicals can be added to complex contaminant metals such Cu²⁺, which can also bind with ferrozine and yield falsely elevated iron values. See Ceriotti et al., Improved direct specific determination of serum iron and total iron-binding capacity Clin Chem. 1980, 26(2), 327-31, the contents of which are incorporated herein by reference.
Inductively Coupled Plasma Atomic Emission Spectroscopy (ICP-AES)—is an emission spectroscopy technique that quantifies the mass percentage of metals in a sample. ICP-AES is based on the excitation of metal atoms/ions in the sample using a plasma (an ionized gas consisting of positive ions and free electrons) and analyzing the emission wavelength of the electromagnetic radiation, which is typical of that particular metal. Even though the technique is a standard analytical technique within common general knowledge of the skilled person, further information on ICP-AES can be found in Manley et al., Simultaneous Cu-, Fe-, and Zn-specific detection of metalloproteins contained in rabbit plasma by size-exclusion chromatography-inductively coupled plasma atomic emission spectroscopy. J Biol Inorg Chem. 2009, 14, 61-74, the contents of which are incorporated herein by reference.

The preferred method of determining the iron content of a sample for the purposes of the therapeutic method of the present invention is ICP-AES. The iron saturation of transferrin is then calculated based on the transferrin protein concentration, total iron content of the sample, and the fact that wild type transferrin has two iron-binding sites. Wild type human transferrin (molecular weight 79,750) can bind two iron atoms such that a sample containing 1 g of transferrin would be 100% saturated by 1.4 mg of iron.

Where the transferrin concentration of a particular sample is unknown it can be readily determined by a variety of well-characterized immunological (ELISA, nephelometry) and non-immunological methods (absorbance, AU480 chemical analysis).

At the time of writing, transferrin has not been authorized as a pharmaceutical in any major jurisdiction worldwide. As such, pharmacopoeial monographs do not exist for transferrin. Further information on the physical properties of transferrin, such as iron saturation can be obtained from the main reference text consulted by the skilled person; See L von Bonsdorff et al., Transferrin, Ch 21, pg 301-310, Production of Plasma Proteins for Therapeutic Use, Eds. J. Bertolini et al., Wiley, 2013 [Print ISBN:9780470924310 |Online ISBN:9781118356807], the contents of which are incorporated herein by reference and would be deemed to be within the common general knowledge of the skilled person.

By “lactoferrin” the current specification is to be construed as meaning a therapeutically effective amount of:
a wild type (mammalian, preferably human) lactoferrin protein,
a functional mutant thereof,
a functional fragment thereof, or
combinations thereof.

The iron saturation of the lactoferrin, functional mutant thereof, or functional fragment thereof may be about 50% or less. Preferably, the iron saturation is about 40% or less. In one embodiment, the iron saturation is about 30% or less. For example, the iron saturation may be about 20% or less, such as about 10% or less. In some embodiments, the iron saturation is about 5% or less. In yet a further embodiment, the iron saturation may be less than about 1%.

As used herein, “apo-lactoferrin” shall mean lactoferrin having an iron saturation of less than 1%. Similarly, “holo-lactoferrin” shall mean lactoferrin having an iron saturation of 99% or greater. Iron content, and saturation levels for lactoferrin can be measured analogously to those of transferrin discussed in detail above.

In using the terms transferrin and lactoferrin the present specification includes within its scope recombinant derivatives of transferrin and lactoferrin that differ from the wild type amino acid sequences of the human proteins, outlined in SEQ ID NOS: 1 & 2 respectively, by one or more substitutions, one or more deletions, or one or more insertions that may not materially alter the structure, or hydropathic nature of the recombinant proteins relative to the wild type proteins. Recombinant variants of transferrin and lactoferrin within the scope of the present invention may additionally comprise at least one post translational modification, such as pegylation, glycosylation, polysialylation, or combinations thereof.

In one embodiment, the present invention contemplates recombinant variants of transferrin and lactoferrin having one or more conservative substitutions relative to the wild type proteins in SEQ ID NOS: 1 & 2. A “conservative substitution” is one in which an amino acid is substituted for another amino acid that has similar properties, such that one skilled in the art of peptide chemistry would expect the secondary structure and hydropathic nature of the polypeptide to be substantially unchanged. Generally, change within the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

For example, the recombinant transferrin or lactoferrin within the scope of the method of treatment of the present invention may possess at least 90%, 95%, 96%, 97%, 98% or 99% homology with the wild type human transferrin and human lactoferrin proteins outlined in SEQ ID NO: 1 and SEQ ID NO: 2 respectively.

In a further embodiment, the present invention includes specific mutant forms of transferrin and/or lactoferrin that maintain their structure but prevent the protein binding iron to one or the other of the iron-biding domains, e.g. the N-lobe, the C-lobe, or a combination thereof.

Transferrin mutants within the scope of the present invention include, but are not limited to:
i) Y188F mutant N lobe (SEQ ID NO: 3);
ii) Y95F/Y188F mutant N lobe (SEQ ID NO: 4); and
iii) Y426F/Y517F mutant C lobe (SEQ ID NO: 5).

The skilled person will appreciate that recombinant proteins can be obtained utilising standard techniques well known in the art of protein expression, production and purification. Nucleic acid sequences of a recombinant protein of interest can be inserted in any expression vector suitable for expression in the elected host cell, e.g. mammalian cells, insect cells, plant cells, yeast, and bacteria.

As used herein, the term “expression vector” refers to an entity capable of introducing a protein expression construct into a host cell. Some expression vectors also replicate inside host cells, which increases protein expression by the protein expression construct. One type of vector is a “plasmid,” which refers to a circular double stranded DNA loop into which additional DNA segments may be ligated. Other vectors include cosmids, bacterial artificial chromosomes (BAC) and yeast artificial chromosomes (YAC), fosmids, phage and phagemids. Another type of vector is a viral vector, wherein additional DNA segments may be ligated into the viral genome. Certain vectors are capable of autonomous replication in a host cell into which they are introduced (e.g., vectors having an origin of replication which functions in the host cell). Other vectors can be integrated into the genome of a host cell upon introduction into the host cell and are thereby replicated along with the host genome. Moreover, certain preferred vectors are capable of directing the expression of genes to which they are operatively linked.

Suitable bacterial cells include Escherichia coli, Bacillus subtilis, Salmonella typhimurium, Pseudomonas spp., Streptomyces spp., and Staphylococcus spp. Suitable yeast cells include Saccharomyces spp., Pichia spp., and Kuyveromyces spp. Suitable insect cells include those derived from Bombyx mori, Mamestra brassicae, Spodoptera frugiperda, Trichoplusia ni and Drosophila melanogaster. Such mammalian host cells include but are not limited to CHO, VERO, BHK, Hela, COS, MDCK, W138, BT483, Hs578T, HTB2, BT2O and 147D, NSO, CRL7O3O, HsS78Bst, human hepatocellular carcinoma cells (e.g. Hep G2), human adenovirus transformed 293 cells (e.g. HEK293), PER.C6, mouse L-929 cells, HaK hamster cell lines, murine 313 cells derived from Swiss, Balb-c or NIH mice, and CV-1 cell line cells.

The present invention also contemplates the use of wild type and recombinant transferrin and lactoferrin proteins that are conjugated or fused to any other protein, protein fragment, protein domain, peptide, small molecule or other chemical entity. For example, suitable fusion or conjugation partners include serum albumins (for example, bovine, rabbit or human), keyhole limpet hemocyanin, immunoglobulin molecules (including the Fc domain of immunoglobulins), thyroglobulin, ovalbumin, tetanus toxoid, or a toxoid from other pathogenic bacteria, or an attenuated toxin derivative, cytokines, chemokines, glucagon-like peptide-1, exendin-4, XTEN, or combinations thereof.

In one embodiment of the invention, the transferrin and lactoferrin proteins used in the method of the present invention are fusion proteins having an improved in-vivo half-life, in which:
the wild type (mammalian, preferably human) transferrin or lactoferrin protein is fused to a fusion partner selected from an immunoglobulin Fc domain and albumin; or
a mutant transferrin or lactoferrin protein within the scope of the method of the present invention is fused to a fusion partner selected from an immunoglobulin Fc domain and albumin.

In one embodiment, the preferred fusion partner is an immunoglobulin Fc domain. For example, the immunoglobulin Fc domain may comprise at least a portion of a constant heavy immunoglobulin domain. The constant heavy immunoglobulin domain is preferably an Fc fragment comprising the CH2 and CH3 domain and, optionally, at least a part of the hinge region. The immunoglobulin Fc domain may be an IgG, IgM, IgD, IgA or IgE immunoglobulin Fc domain, or a modified immunoglobulin Fc domain derived therefrom. Preferably, the immunoglobulin Fc domain comprises at least a portion of a constant IgG immunoglobulin Fc domain. The IgG immunoglobulin Fc domain may be selected from IgG1, IgG2, IgG3 or IgG4 Fc domains, or modified Fc domains thereof.

In one embodiment, the fusion protein may comprise transferrin fused to an IgG1 Fc domain. In one embodiment, the fusion protein may comprise a transferrin mutant fused to an IgG1 Fc domain.

Neurodegenerative Event

Surprisingly, the present inventors have discovered that transferrin and lactoferrin have an unexpected therapeutic role outside of iron binding/delivery to cells, in that both proteins were highly effective in stimulating the development of neural cells from neural progenitor cells and/or neural stem cells. The present invention thus provides for a method of stimulating neural cell development in a patient that has suffered a neurodegenerative event, the method comprising administering a therapeutically effective amount of a protein selected from transferrin, lactoferrin, and combinations thereof to the patient in need thereof.

As used herein, the term “stimulating neural cell development” is utilised to mean that the transferrin or lactoferrin are having a direct or indirect effect on neural progenitor cells and/or neural stem cells in the patient so as to produce new neural cells. Without wishing to limit the generality of the invention, it is postulated that the administration of transferrin or lactoferrin results in an increase in at least one of:
i) proliferation of the neural progenitor cells and/or neural stem cells within the patient, or
ii) inducing differentiation of the neural progenitor cells and/or neural stem cells into differentiated neural cells, compared to neural progenitor cells/neural stem cells that have not been exposed to the transferrin, or lactoferrin.

By “neural cells”, the present specification includes all cells of the nervous system including without limitation glial cells, and neuronal cells. In one embodiment, the neural cells referred to in the method of the present invention are neuronal cells and the transferrin and lactoferrin potentiate the neurogenesis of new neuronal cells.

As used herein, the term “neurodegenerative event” refers to an event that causes the loss of structure and/or function of neural cells and includes the death of neural cells. The event may be an isolated one-off event/occurrence causing immediate neural cell damage or death. Alternatively, the event may be a continuous or chronic event that progressively leads to increasing levels of neural cell damage or death. In a particular embodiment, the neurodegenerative event causes the loss of structure, loss of function, or death of neuronal cells (or neurons) in the brain and/or spinal cord resulting in brain and/or spinal cord damage and dysfunction.

In one embodiment, the neurodegenerative event is caused by a neurodegenerative disease. By “neurodegenerative disease” is meant any disease characterized by the dysfunction and/or death of neurons leading to a loss of neurologic function in the brain, spinal cord, central nervous system, and/or peripheral nervous system. Neurodegenerative diseases within the scope of the present invention can be chronic or acute.

Non-limiting examples of neurodegenerative diseases within the scope of the present invention include Parkinson's disease, frontotemporal dementia, Alzheimer's disease, Mild Cognitive Impairment, Diffuse Lewy body disease, Dementia with Lewy bodies type, demyelinating diseases such as multiple sclerosis and acute transverse myelitis, amyotrophic lateral sclerosis, Huntington's disease, Creutzfeldt-Jakob disease, corticobasal ganglionic degeneration, peripheral neuropathy, progressive supranuclear Palsy, spinocerebellar degenerations, spinal ataxia, Friedreich's ataxia, cerebellar cortical degenerations, neurogenic muscular atrophies, anterior horn cell degeneration, infantile spinal muscular atrophy, and juvenile spinal muscular atrophy, subacute sclerosing panencephalitis, Hallervorden-Spatz disease, dementia pugilistica, Pick's disease, tauopathies, synucleinopathies, and combinations thereof.

In one embodiment, the neurodegenerative disease may be selected from the group consisting of Parkinson's disease, Alzheimer's disease, multiple sclerosis, amyotrophic lateral sclerosis, and Huntington's disease. For example, the neurodegenerative disease may be Parkinson's disease.

As a non-limiting/non-binding theory, it is known that neurodegenerative insult or injury causes neural stem cells to migrate to the site of such insult or injury. See Arvidsson et al., 2002, Nat. Med., 8, 963-970; Kokaia and Lindvall, 2003, Curr. Opin. Neurobiol., 13, 127-132; and Kernie et al., 2010, Neurobiol. Disease, 37, 267-274. The present inventors postulate that by increasing the concentration of transferrin, lactoferrin, or combinations thereof within the patient such molecules can potentiate and/or promote the body's own neuroregenerative repair mechanisms. Transferrin and lactoferrin can be administered directly or indirectly to the site of neurodegenerative insult or injury by any conventional drug delivery means known by those skilled in the art.

It should be appreciated by those skilled in the art that the specific embodiments disclosed within above paragraphs should not be read in isolation, and that the present specification intends for these embodiments to be disclosed in combination with other embodiments as opposed to being disclosed individually. For example, each of the embodiments disclosed is to be read as being explicitly combined with each of the embodiments, or any permutation of 2 or more of the embodiments disclosed therein.

Combination Therapy

The method of the present invention also contemplates the use of supplementary active compounds and molecules in combination with transferrin and/or lactoferrin.

The supplementary active compounds and molecules can be co-formulated with transferrin or lactoferrin as a unit dosage form, i.e. as a physically discrete unit intended as a unitary dosage for the subject to be treated. Alternatively, the supplementary active compounds and molecules can be presented as a kit-of-parts, and:
administered separately to transferrin and/or lactoferrin, in a phased or sequential dosing pattern; or
co-administered simultaneously from different dosage forms.

For example, the method of the present invention contemplates administering other serum, or plasma-based proteins in combination with transferrin and/or lactoferrin. Serum or plasma proteins within the scope of the present invention include those purified from a suitable plasma source, such as human plasma, and those prepared using recombinant manufacturing techniques. For example, the serum or plasma protein may be selected from the group consisting of Albumin (e.g. ALBUTEIN), Alpha-1 Antitrypsin/Alpha-1 Proteinase Inhibitor (e.g. PROLASTIN), Antithrombin (e.g. THROMBATE III), polyclonal immunoglobulins (IgG, IgA, and combinations thereof), polyspecific immunoglobulins (IgM), C1 esterase inhibitor (e.g. BERINERT), Transthyretin, and combinations thereof.

Exemplary polyclonal immunoglobulins within the scope of the present invention include commercially available polyclonal IgG formulations such as FLEBOGAMMA DIF 5% & 10%, GAMUNEX-C 10%, BIVIGAM 10%, GAMMAGARD Liquid 10%, etc.

Exemplary polyspecific immunoglobulins (IgM) within the scope of the present invention include commercially available immunoglobulin formulations containing polyspecific IgM such as PENTAGLOBIN or TRIMODULIN.

In one embodiment, the serum or plasma protein may be selected from the group consisting of Albumin, Antithrombin, Alpha-1 Antitrypsin, C1 esterase inhibitor, and combinations thereof. For example, the serum or plasma protein may be selected from the group consisting of Antithrombin, Alpha-1 Antitrypsin, and combinations thereof. In a particular embodiment, a therapeutically effective amount of Alpha-1 Antitrypsin is administered to the patient in addition to the protein selected from transferrin, lactoferrin, and combinations thereof. In a particular embodiment, a therapeutically effective amount of Antithrombin is administered to the patient in addition to the protein selected from transferrin, lactoferrin, and combinations thereof.

The method of the present invention also provides for administering known neurogenic/neurotrophic compounds and molecules in combination with transferrin and/or lactoferrin. For example, the method of the present invention contemplates administering neurogenic/neurotrophic proteins, peptides, and small molecules alongside transferrin and/or lactoferrin.

Suitable neurogenic and/or neurotrophic compounds and molecules may be selected from the group consisting of BDNF (brain-derived neurotrophic factor; NGF superfamily; SEQ ID NO: 6), GNDF (glial cell line-derived neurotrophic factor; TGF-β superfamily; SEQ ID NO: 7), CNTF (cilliary neurotrophic factor-1; neurokine superfamily; SEQ ID NO: 8), PACAP (amino acids 1-38 of pituitary adenylate cyclase-activating polypeptide; SEQ ID NO: 9), Y-27632 and pharmaceutically acceptable salts thereof [trans-4-[(1R)-1-Aminoethyl]-N-4-pyridinylcyclohexanecarboxamide], Fasudil and pharmaceutically acceptable salts thereof [hexahydro-1-(5-isoquinolinyl-sulfonyl)-1H-1,4-diazepine], and combinations thereof.

The skilled person will appreciate that the present invention also contemplates within its scope covalent conjugates of each of the above listed compounds and molecules to each of transferrin and lactoferrin. Furthermore, it will be appreciated that the present invention also contemplates within its scope recombinant fusion proteins of each of the above listed proteins and peptides with each of transferrin and lactoferrin.

In one embodiment, the transferrin, lactoferrin, or combinations thereof may constitute at least 20% by weight of the total protein content utilised in the therapeutic method of the present invention. For example, the transferrin, lactoferrin, or combinations thereof may constitute greater than or equal to about 30%, 40%, 50%, 60%, 70%, 75%, 80%, 85%, 90%, 93%, 95%, 96%, 97%, 98%, or 99% by weight of the total protein content utilised in the combination therapy of the present invention.

It should be appreciated by those skilled in the art that the specific embodiments disclosed within above paragraphs should not be read in isolation, and that the present specification intends for these embodiments to be disclosed in combination with other embodiments as opposed to being disclosed individually. For example, each of the embodiments disclosed is to be read as being explicitly combined with each of the embodiments, or any permutation of 2 or more of the embodiments disclosed therein.

Pharmaceutical Compositions of the Invention

In a further aspect, the present invention also provides for a pharmaceutical composition comprising transferrin, lactoferrin, or combinations thereof for use in the generation of new neural cells in a patient that has suffered a neurodegenerative event.

The pharmaceutical compositions of the present invention may optionally further comprise at least one pharmaceutically acceptable carrier. The at least one pharmaceutically acceptable carrier may be chosen from adjuvants and vehicles. The at least one pharmaceutically acceptable carrier, includes any and all solvents, diluents, other liquid vehicles, dispersion aids, suspension aids, surface active agents, isotonic agents, thickening agents, emulsifying agents, preservatives, as suited to the particular dosage form desired.

Suitable carriers are described in Remington: The Science and Practice of Pharmacy, 21 st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York, the contents of which are incorporated herein by reference. Preferred examples of such carriers or diluents include, but are not limited to, water, saline, Ringer's solutions, glycols, dextrose solution, buffered solutions (such as phosphates, glycine, sorbic acid, and potassium sorbate) and 5% human serum albumin. Liposomes and non-aqueous vehicles such as glyceride mixtures of saturated vegetable fatty acids, and fixed oils (such as peanut oil, cottonseed oil, safflower oil, sesame oil, olive oil, corn oil and soybean oil) may also be used depending on the route of administration.

The pharmaceutical composition of the invention is formulated to be compatible with its intended route of administration. For systemic use, the pharmaceutical composition of the invention can be formulated for administration by a conventional route selected from the group consisting of intravenous, subcutaneous, intramuscular, intradermal, intraperitoneal, intracerebral, intracranial, intrapulmonary, intranasal, intraspinal, intrathecal, transdermal, transmucosal, oral, vaginal, and rectal.

In one embodiment, parenteral administration is the preferred route of administration. The pharmaceutical composition may be enclosed in ampoules, disposable syringes, sealed bags, or multiple dose vials made of glass or plastic. In one embodiment, administration as an intravenous injection is the preferred route of administration. The formulations can be administered continuously by infusion or by bolus injection.

The pharmaceutical compositions of the present invention may be presented as a unit dosage unit form, i.e. as physically discrete units intended as unitary dosages for the subject to be treated.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, CREMOPHOR EL, or phosphate buffered saline (PBS). In all cases, the composition must be sterile and should be fluid to the extent that easy syringe-ability exists.

The compositions of the invention should be stable under the conditions of manufacture and storage. Moreover, compositions should be preserved against the contaminating action of microorganisms such as bacteria and fungi. The carrier can be a solvent or dispersion medium containing, for example water, ethanol, polyols (for example, glycerol, propylene glycol, and liquid polyethylene glycol, and the like), and suitable mixtures thereof.

Prevention of the action of microorganisms can be achieved by various antibacterial and antifungal agents, for example parabens, chlorobutanol, phenol, ascorbic acid, thimerosal, and the like. In many cases, it will be preferable to include isotonic agents, for example sugars (such as mannitol, sorbitol, etc.), polyalcohols, or sodium chloride in the composition. Prolonged absorption of the injectable compositions can be brought about by including in the composition an agent which delays absorption, for example aluminum monostearate or gelatin.

Sterile injectable solutions of the pharmaceutical composition of the present invention can be prepared by incorporating the active molecule in the required amount in an appropriate solvent with one or a combination of ingredients as discussed above followed by filtered sterilization. In the case of sterile powders for the preparation of sterile injectable solutions, methods of preparation include vacuum drying and freeze-drying that provide a powder of the active ingredient plus any additional desired ingredient from a previously sterile-filtered solution thereof.

Except insofar as any conventional media or agent is incompatible with the active molecules of the present invention use thereof in the compositions is contemplated to be within the scope of the present invention.

In one embodiment, the transferrin, lactoferrin, or combinations thereof may constitute at least 20% by weight of the total protein content of the pharmaceutical composition of the present invention. For example, the transferrin, lactoferrin, or combinations thereof may constitute greater than or equal to about 30%, 40%, 50%, 60%, 70%, 75%, 80%, 85%, 90%, 93%, 95%, 96%, 97%, 98%, or 99% by weight of the total protein content of the pharmaceutical composition of the present invention.

It should be appreciated by those skilled in the art that the specific embodiments disclosed within above paragraphs should not be read in isolation, and that the present specification intends for these embodiments to be disclosed in combination with other embodiments as opposed to being disclosed individually. For example, each of the embodiments disclosed is to be read as being explicitly combined with each of the embodiments, or any permutation of 2 or more of the embodiments disclosed therein.

Dosing

As discussed supra, the present inventors postulate that by increasing the concentration of transferrin, lactoferrin, or combinations thereof proximate to the site of a neurodegenerative insult or injury, such molecules can potentiate and/or promote the body's own neuroregenerative repair mechanisms. Transferrin and lactoferrin could be administered directly or indirectly to the site of neurodegenerative insult or injury by any conventional drug delivery means known by those skilled in the art. For example, the transferrin, lactoferrin, or combinations thereof could be administered locally or proximate to the injury caused by the neurodegenerative event by a conventional route selected from the group consisting of intracerebral, intracranial, intraspinal, and intrathecal. For example, the transferrin, lactoferrin, and combinations thereof may be administered locally during surgical intervention.

Alternatively, the transferrin, lactoferrin, or combinations thereof could be delivered indirectly to the site of the neurodegenerative insult or injury by an administration route selected from the group consisting of intravenous, subcutaneous, intramuscular, intradermal, intraperitoneal, intrapulmonary, intranasal, transdermal, transmucosal, oral, vaginal and rectal.

For the avoidance of any doubt, the opportunity is taken to clarify that the present specification speaks to transferrin iron saturation levels in two separate and distinct contexts:
a) In the first context, as outlined, the specification is speaking to the iron saturation of purified exogenous transferrin in a pharmaceutical composition that is to be administered to a patient. In this instance, iron saturation levels of the purified exogenous transferrin can be determined using Inductively Coupled Plasma Atomic Emission Spectroscopy (but other methods, such as colorimetric methods can also be used).
b) In the second context, which will be discussed in more detail immediately below, the present specification is speaking to measuring the iron saturation of physiological transferrin in a patient, i.e. in the patient's plasma or serum, after the pharmaceutical composition containing exogenous transferrin has been administered to the patient.

Under normal physiological conditions, practically all iron in plasma is bound to transferrin and the resulting iron saturation of physiological transferrin is approximately 30%. In Example 6 (vide infra), the present inventors have demonstrated that transferrin with an iron saturation of less than 30% results in an unexpected neuroregenerative effect. As a non-limiting hypothesis, it is envisaged that by administering a pharmaceutical composition containing exogenous transferrin (with a low iron saturation) to a patient that the physiological concentrations of transferrin within the patient's plasma will increase resulting in the iron saturation of physiological transferrin dropping below 30%. Thus, allowing physiological transferrin to leverage a neuroregenerative effect. Naturally, exogenous transferrin with an iron saturation of less than 1% will likely be more efficacious than exogenous transferrin with an iron saturation 40%.

Accordingly, in one embodiment, the protein selected from transferrin, lactoferrin, and combinations thereof is administered to the patient at a concentration sufficient to reduce the iron saturation of the patient's transferrin (in a serum or plasma sample of the patient) below about 30%. Preferably, the protein selected from transferrin, lactoferrin, and combinations thereof is administered to the patient at a concentration sufficient to reduce the iron saturation of the patient's transferrin (in a serum or plasma sample of the patient) below about 20%, for example below about 10%. The transferrin, lactoferrin, or combinations thereof may be administered to the patient using a titration based-dosage regimen to achieve this level of serum or plasma transferrin iron saturation.

The skilled person will appreciate that the measurement of transferrin iron saturation levels in a patient's serum or plasma is a routine assay typically performed using colorimetric methodologies as discussed supra. Plasma or serum iron content, is measured on chemical analyzers by using a colorimetric reaction with ferene or ferrozine as a chromogen to form a colour complex with iron. An analysed sample produces two values:
sample iron content (i.e. iron bound to transferrin in the sample), and unsaturated iron binding capacity (UIBC, i.e. the number of unoccupied iron biding sites on transferrin in the sample).
Total iron binding capacity (TIBC) is the sum of the sample iron content and UIBC.
Transferrin saturation (%) is determined as [(sample iron content/TIBC)×100].

The workings of colorimetric assays for the measurement of transferrin iron saturation levels in a patient's serum or plasma are common general knowledge and further information can be found in various literature reviews, such as Pfeiffer et al., Am J Clin Nutr 2017, 106(Suppl), 1606S-14S, the contents of which are incorporated herein by reference.

In yet a further embodiment of the method of the present invention the protein selected from transferrin, lactoferrin, and combinations thereof can be administered to the patient at a concentration of from about 5 mg/kg to about 8400 mg/kg. For example, from about 10 mg/kg to about 7000 mg/kg, such as from about 20 mg/kg to about 6000 mg/kg, for example from about 50 mg/kg to about 5000 mg/kg. In some embodiments the protein selected from transferrin, lactoferrin, and combinations thereof can be administered to the patient at a concentration of from about 50 mg/kg to about 1000 mg/kg. Suitably, the protein can be administered at a concentration of from about 50 mg/kg to about 500 mg/kg, such as from about 50 mg/kg to about 250 mg/kg, for example from about 50 mg/kg to about 150 mg/kg.

In one embodiment, the method of the present invention may comprise administering the protein selected from transferrin, lactoferrin, and combinations thereof to a patient in need thereof as part of a multiple dosing regimen. For example, at initial dose of about 50 mg/kg to about 5000 mg/kg on day 1 of an administration period, followed by about 50 mg/kg to about 1000 mg/kg per dose during a multiple dosing period. For example, at initial dose of about 50 mg/kg to about 1000 mg/kg on day 1 of an administration period, followed by about 50 mg/kg to about 500 mg/kg per dose during a multiple dosing period. For example, at initial dose of about 50 mg/kg to about 500 mg/kg on day 1 of an administration period, followed by about 50 mg/kg to about 250 mg/kg per dose during a multiple dosing period. For example, at initial dose of about 50 mg/kg to about 250 mg/kg on day 1 of an administration period, followed by about 50 mg/kg to about 250 mg/kg per dose during a multiple dosing period. The multiple dosing period may comprise from about 3 to about 30 administrations up to a total cumulative dose. The multiple dosing period may be from about 1 to about 30 weeks. The multiple portion doses may be administered at intervals of from about 1 day to about 30 days.

It should be appreciated by those skilled in the art that the specific embodiments disclosed within above paragraphs should not be read in isolation, and that the present specification intends for these embodiments to be disclosed in combination with other embodiments as opposed to being disclosed individually. For example, each of the embodiments disclosed is to be read as being explicitly combined with each of the embodiments, or any permutation of 2 or more of the embodiments disclosed therein.

### BRIEF DESCRIPTION OF THE DRAWINGS

Additional features and advantages of the present invention will be made clearer in the appended drawings, in which:

FIGS. 1A to 1D show the induction of neurite outgrowth, and proliferation in SH-SYSY cells and increased β-III-tubulin protein concentrations in response to apo-transferrin;

FIGS. 2A to 2B demonstrate that apo-transferrin induces primary human neural progenitor cells to become β-III-tubulin protein positive neurons and GFAP protein positive astrocytes cells;

FIG. 3A plots the effect of deferoxamine mesylate at various concentrations relative to apo-transferrin on neurite outgrowth in SH-SYSY cells;

FIG. 3B illustrates the efficacy of a transferrin mutant having reduced iron binding capacity on promoting neurite outgrowth in SH-SYSY cells;

FIG. 4 plots the effect of various different proteins on neurite outgrowth in SH-SYSY cells;

FIG. 5 plots the effect of IOX2, a prolyl hydroxylase inhibitor, on neurite outgrowth in SH-SYSY cells;

FIGS. 6A & 6B illustrate the role of iron saturation on the efficacy of transferrin in promoting neurite outgrowth in SH-SY5Y cells;

FIGS. 7A to 7D plots the effect of apo-transferrin in combination with other neurotrophic proteins/peptide fragments on neurite outgrowth in SH-SY5Y cells;

FIG. 8 plots the effect of apo-transferrin in combination with the small molecule Y-27632 on neurite outgrowth in SH-SY5Y cells; and

FIG. 9 demonstrates the positive regenerative effect of apo-transferrin in MPTP-induced Parkinson's Disease in mice.

### DETAILED EXAMPLES OF THE INVENTION

It should be readily apparent to one of ordinary skill in the art that the examples disclosed herein below represent generalised examples only, and that other arrangements and methods capable of reproducing the invention are possible and are embraced by the present invention.

### Example 1: Apo-Transferrin (ApoTf) Induces Differentiation and Neurite Outgrowth in SH-SY5Y Cells in a Dose Responsive Manner

Transferrin is utilized in cell culture and in-vivo to deliver iron as a nutrient to cells. This is typically accomplished through the actions of holo-transferrin (HoloTf) binding to, and endocytosis by, its cognate receptor CD71, the transferrin receptor 1 (TfR1). Transferrin is typically believed to provide cells with iron as a means to promote and sustain metabolic activity. The present inventors have surprisingly found that apo-transferrin, the iron-free form of transferrin protein, induces differentiation of a very common research model of neurons, SH-SY5Y cells. Induction of neuronal differentiation was assessed by morphological parameters of neurite formation (a key 30 element typically used as a marker of neuronal differentiation, neuronal health, and function) according to the procedures of Agholme, 2010. J. of Alzheimer's Disease. Vol. 20:1p 069-108; and Dyberg et al., 2017. PNAS Vol 114 (32), E6603-E6612.

Undifferentiated SH-SY5Y cells were seeded into 96 well clear bottom plates in media containing 0.1% FBS. A serum-free base media was utilized as recommended by the supplier for SH-SY5Y cells (Sigma, Cat #94030304-1VL). Twenty-four hours after seeding cells, a 3× stock solution of ApoTf, final concentrations indicated on the x-axis, in serum free base media was added to the cells. ApoTf was obtained and purified from pooled human plasma and dosed at a final concentration of 0.2 mg/mL. Cells were allowed to differentiate for 6 days. Neurite growth was assessed by imaging and image analysis. At the time of analysis, a 10× solution of Tubulin Tracker (Molecular Probes, T34075) and Hoechst 33342 (Molecular Probes #H3570) nuclear stain was prepared.

Briefly, Tubulin Tracker dissolved in DMSO was diluted 1:1 with Pluronic F-127 and further diluted into HBSS to generate a 10× solution. Hoechst 33342 was added to the HBSS-Tubulin Tracker solution at 10 μg/mL to generate a 10× nuclear stain. The 10× staining solution (10 μL) was added directly to treated assay wells and incubated at 37° C. for 30 minutes. Following incubation, 110 μL of 0.4% Trypan Blue was added directly to assay wells and imaged on a Molecular Devices Nano imaging instrument. Nine images/well were acquired in the blue (Nuclei) and green (Tubulin) fluorescent channels for each image.

After obtaining images, the MetaExpress Neurite Outgrowth analysis module (Molecular Devices) was used to identify cells, cell bodies, and quantify neurites. The total number of neurite branches were divided by the total number of cells imaged to account for different numbers of cells in each test well. The Outgrowth Fold Change was determined by setting the untreated control cells to a value of 1 with all other treatments shown relative to untreated control.

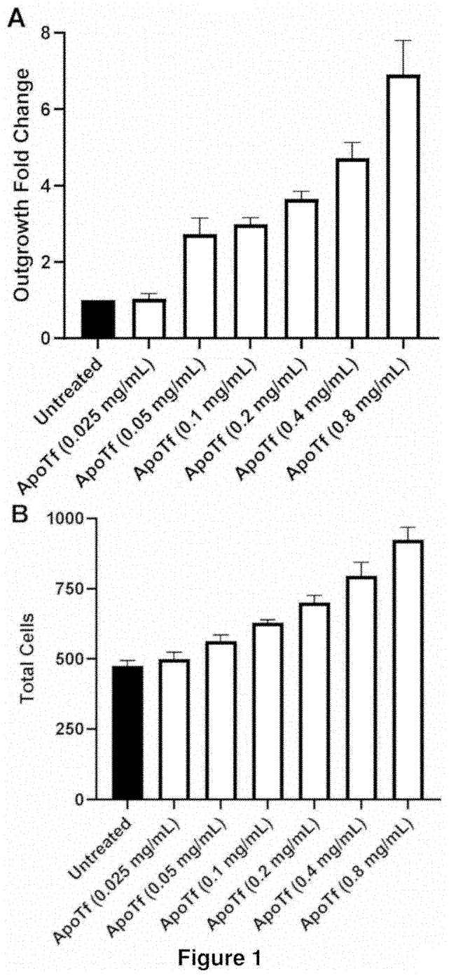

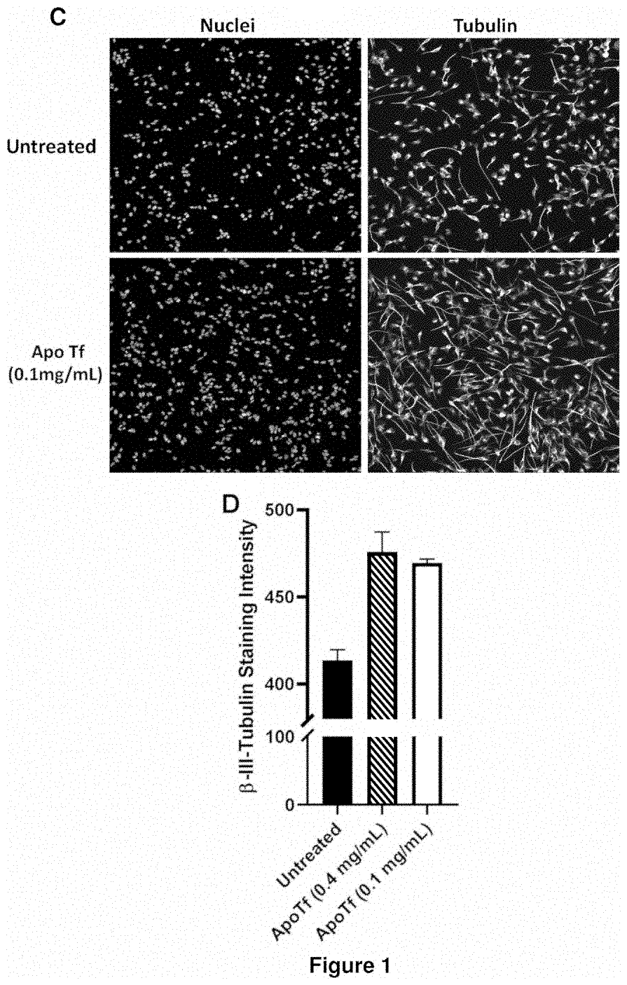

From FIG. 1A it is evident that apo-transferrin was able to induce neurite outgrowth in a dose dependent manner. Incremental increases in apo-transferrin concentration, up to a maximum of 0.8 mg/mL, were associated with an improved outgrowth response in the SH-SY5Y cells. This phenomenon is counterintuitive to the known function of transferrin, which primarily acts in the holo- or iron-laden form of transferrin.

FIG. 1B illustrates that apo-transferrin induces a concentration dependent increase in cell numbers. Increased cell number is indicative of increased cell proliferation, up to the maximum tested dose of 0.8 mg/mL apo-transferrin.

FIG. 1C provides visual comparison of SH-SY5Y cells treated with 0.1 mg/mL ApoTf (lower panels) to an untreated control (upper panels). Left panels show nuclear staining with Hoechst 33342. Right images show tubulin staining of cell bodies and neurites. From FIG. 1C it is apparent that ApoTf had a profound effect on promoting cell proliferation, and subsequently/simultaneously promoting induction of neurite/tubulin outgrowth.

Additionally, as shown in FIG. 1D, it was found that apo-transferrin treatment caused an increase in β-III-tubulin protein, a well-characterized, traditional marker of neurons. In this experiment, the SH-SY5Y cells were differentiated as described supra. At the time of analysis, cells were fixed with paraformaldehyde, stained for β-III-tubulin (R&D Systems, MAB1195), and imaged on a Molecular Devices Nano imaging instrument. Image analysis was performed by assessing the fluorescent intensity of cells stained with β-III-tubulin. Background from secondary antibody alone was subtracted from all values. Values are shown with standard deviations as “β-III-Tubulin Staining Intensity” for the indicated conditions.

SH-SY5Y Cells

By “SH-SY5Y cells” the present specification means a subcloned cell line derived from the SK-N-SH neuroblastoma cell line. It serves as a model for neurodegenerative disorders since the cells can be converted to various types of functional neural cells by the addition of specific compounds. In addition, the SH-SY5Y cell line has been used widely in experimental neurological studies, including analysis of neuronal differentiation, metabolism, and function related to neurodegenerative processes, neurotoxicity, and neuroprotection.

Outlined herein under are peer reviewed citations referencing the SH-SY5Y cell line as a predictive model for various neurodegenerative disorders. The list does not constitute an admission of prior art by the inventors, rather it serves to illustrate the skilled person's knowledge of the utility of the SH-SY5Y cell line as a predictive model for neurodegenerative disorders.

Neurogenesis
Dayem et al. Biologically synthesized silver nanoparticles induce neuronal differentiation of SH-SY5Y cells via modulation of reactive oxygen species, phosphatases, and kinase signaling pathways. Biotechnol. J. 2014, 9, 934-943.
Fagerstrom et al. Protein Kinase C-epsilon Implicated in Neurite Outgrowth in Differentiating Human Neuroblastoma Cells. Cell Growth & Differentiation Vol. 7, 775-785, June 1996.

Mood Stabilization (Depression)
Yuan et al. The Mood Stabilizer Valproic Acid Activates Mitogen-activated Protein Kinases and Promotes Neurite Growth. JBC Vol. 276, No. 34, Issue of August 24, pp. 31674-31683, 2001.
Tatro et al. Modulation of Glucocorticoid Receptor Nuclear Translocation in Neurons by Immunophilins FKBP51 and FKBP52: Implications for Major Depressive Disorder. Brain Res. 2009 Aug. 25; 1286: 1-12.
Laifenfeld et al. Norepinephrine alters the expression of genes involved in neuronal sprouting and differentiation: relevance for major depression and antidepressant mechanisms. Journal of Neurochemistry, 2002, 83, 1054-1064.
Cavarec et al. In Vitro Screening for Drug-Induced Depression and/or Suicidal Adverse Effects: A New Toxicogenomic Assay Based on CE-SSCP Analysis of HTR2C mRNA Editing in SH-SY5Y Cells. Neurotoxicity Research. January 2013, Vol. 23 Issue 1, p 49-62.

Tauopathy (Alzheimer's Disease, FTD, and Other Neurodegenerative Diseases with Abnormal Tau)
Jamsa et al. The retinoic acid and brain-derived neurotrophic factor differentiated SH-SY5Y cell line as a model for Alzheimer's disease-like tau phosphorylation. Biochemical and Biophysical Research Communications 319 (2004) 993-1000.
Seidel et. al. Induced Tauopathy in a Novel 3D-Culture Model Mediates Neurodegenerative Processes: A Real-Time Study on Biochips. PLOS One. (November 2012) Volume 7 Issue 11. e49150.
Karch et al. Extracellular Tau Levels Are Influenced by Variability in Tau That Is Associated with Tauopathies. JBC VOL. 287, NO. 51, pp. 42751-42762, Dec. 14, 2012.

Alzheimer's Disease
Pettifer et al. Guanosine protects SH-SY5Ycells against b-amyloid-induced apoptosis. NeuroReport 2004 15(5):833-836.
Tanii et al. Alzheimer's Disease Presenilin-1 Exon 9 Deletion And L250s Mutations Sensitize SH-SYSY Neuroblastoma Cells To Hyperosmotic Stress-Induced Apoptosis. Neuroscience Vol. 95, No. 2, pp. 593-601, 2000.
Li et al. Beta-amyloid induces apoptosis in human-derived neurotypic SH-SYSY cells. Brain Res. 1996 Nov. 4; 738(2):196-204.

ALS and Frontotemporal Dementia
Lee et al. Hexanucleotide Repeats in ALS/FTD Form Length-Dependent RNA Foci, Sequester RNA Binding Proteins, and Are Neurotoxic. Cell Reports 5, 1178-1186, Dec. 12, 2013.
Farg et al. C9ORF72, implicated in amytrophic lateral sclerosis and frontotemporal dementia, regulates endosomal trafficking. Human Molecular Genetics, 2014, Vol. 23, No. 13.
Nonaka et al. Phosphorylated and ubiquitinated TDP-43 pathological inclusions in ALS and FTLD-U are recapitulated in SH-SYSY cells. FEBS Letters 583 (2009) 394-400.

Parkinson's Disease
Xing et al. Protective effects and mechanisms of Ndfipl on SH-SYSY cell apoptosis in an in vitro Parkinson's disease model. Genetics and Molecular Research 15 (2): gmr.15026963.
Jung et al. Rosiglitazone protects human neuroblastoma SH-SYSY cells against MPP+ induced cytotoxicity via inhibition of mitochondrial dysfunction and ROS production. Journal of the Neurological Sciences 253 (2007) 53-60.
Choi et al. Signaling Pathway Analysis of MPP+-treated Human Neuroblastoma SH-SYSY Cells. Biotechnology and Bioprocess Engineering 19: 332-340 (2014).

Friedreich's Ataxia
Palomo et al. Silencing of frataxin gene expression triggers p53-.dependent apoptosis in human neuron-like cells. Human Molecular Genetics, 2011, Vol. 20, No. 14 2807-2822.

Huntington's Disease
Banez-Coronel et al. A Pathogenic Mechanism in Huntington's Disease Involves Small CAG-Repeated RNAs with Neurotoxic Activity. Neuroscience Research Volume 53, Issue 3, November 2005, Pages 241-249.
Vidoni et al. Resveratrol protects neuronal-like cells expressing mutant Huntingtin from dopamine toxicity by rescuing ATG4-mediated autophagosome formation. Neurochemistry International 117 (2018) 174-187.
Vidoni et al. Dopamine exacerbates mutant Huntingtin toxicity via oxidative mediated inhibition of autophagy in SH-SYSY neuroblastoma cells: Beneficial effects of anti-oxidant therapeutics. Neurochemistry International 101 (2016) 132-143.
Olsen et al. Examination of mesenchymal stem cell-mediated RNAi transfer to Huntington's disease affected neuronal cells for reduction of huntingtin. Molecular and Cellular Neuroscience 49 (2012) 271-281.

### Example 2: The Effect of ApoTf on β-III-Tubulin and GFAP Protein Concentrations in Primary Human Neural Progenitor Cells

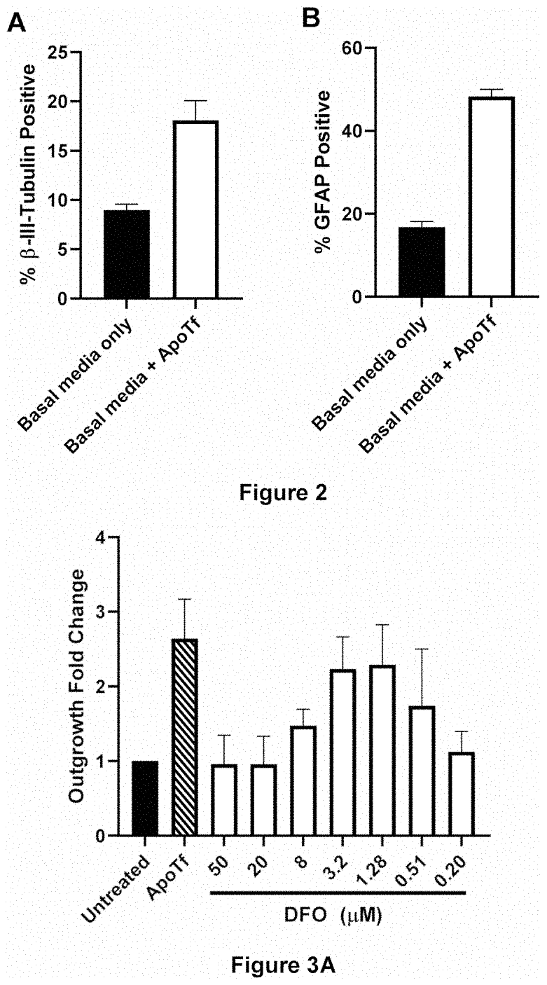

The neurogenic effects of ApoTf also translate to primary human brain cortex-derived neural progenitor cells, another established model of adult neurogenesis (See Azari and Reynolds, “In Vitro Models for Neurogenesis”. Cold Spring Harb Perspect Biol 2016, 8, a021279). As shown in FIGS. 2A and 2B, apo-transferrin dramatically increases the percentage of cells differentiated to neurons (% β-III-tubulin positive cells, 2A) and astrocytes (% GFAP positive cells, 2B), relative to cells without apo-transferrin, from a culture of primary human brain-derived neural progenitor cells.

Neural progenitor cells maintained as neurospheres were obtained from Lonza (PT-2599). Cells were thawed from a frozen vial of neurospheres and cultured in Human NeuroCult™ NS-A Complete Proliferation media (Stemcell Technologies) for 2 weeks. Neurospheres were dissociated to single cells and plated in Laminin coated wells of assay plates. The neural progenitor cells were seeded in NeuroCult™ NS-A Basal media containing 1/10th concentration of the recommended proliferation supplements, in the absence or presence of ApoTf (0.8 mg/mL) for 72 hours. At the time of analysis, cells were fixed with paraformaldehyde, stained for β-III-tubulin (R&D Systems, MAB1195) and GFAP (Invitrogen, PA3-16727), and imaged on a Molecular Devices Nano imaging instrument. Image analysis was performed by assessing the relative numbers of cells staining positive for β-III-tubulin or GFAP. Values for the indicated conditions are shown with standard deviations as “% β-III-Tubulin Positive” cells (FIG. 2A) or “% GFAP Positive” cells (FIG. 2B).

### Example 3: Iron Chelation is not the Sole Mode of Action for Neurogenesis by ApoTf

Deferoxamine mesylate (DFO) is a small molecule iron chelator utilized in clinical practice for iron overload. Like ApoTf, DFO has high affinity binding constants for iron; although only a single iron binding site. The effect of DFO on neurite outgrowth was investigated. ApoTf was tested at a concentration near the bottom of its functional dose curve and compared to DFO's ability to induce neurite outgrowth. ApoTf tested at 2.4 μM (0.2 mg/mL) has two iron binding sites and therefore is comparable to the single iron binding site of DFO at 4.8 μM.

Undifferentiated SH-SYSY cells were seeded and treated as described in Example 1. Neurite growth was assessed by imaging and image analysis. At the time of analysis, a 10× solution of Tubulin Tracker (Molecular Probes, T34075) and Hoechst 33342 (Molecular Probes #H3570) nuclear stain was prepared. Briefly, Tubulin Tracker dissolved in DMSO was diluted 1:1 with Pluronic F-127 and further diluted into HBSS to generate a 10× solution. Hoechst 33342 was added to the HBSS-Tubulin Tracker solution at 10 μg/mL to generate at 10× nuclear stain. The 10× staining solution (10 μL) was added directly to treated assay wells and incubated at 37° C. for 30 minutes. Following incubation, 110 μL of 0.4% Trypan Blue was added directly to assay wells and imaged on a Molecular Devices Nano imaging instrument. Nine images/well were acquired in the blue (Nuclei) and green (Tubulin) fluorescent channels for each image. After obtaining images, the MetaExpress Neurite Outgrowth analysis module (Molecular Devices) was used to identify cell bodies and quantify neurites. The total number of neurite branches were divided by the total number of cells imaged to account for different numbers of cells. The Outgrowth Fold Change was determined by setting the untreated control to a value of 1 with all other treatments shown relative to untreated control. ApoTf was obtained and purified from pooled human plasma and dosed at a final concentration of 0.2 mg/mL. Deferoxamine mesylate (DFO) was obtained from Tocris (Cat #5764), resuspended and stored by the manufacturer's recommendations. Concentrations of DFO that were assessed for neurogenic properties are indicated on the x-axis.

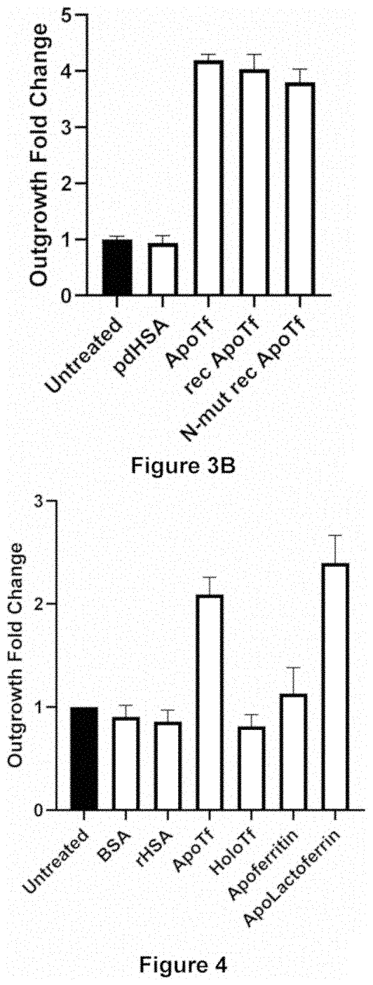

From FIG. 3A it can been seen that DFO shows maximal neurite outgrowth between 1-3 μM, with little neurite formation beyond that concentration, whereas ApoTf continues to increase differentiation even up to 9.9 μM (0.8 mg/mL; 20 μM iron binding sites). These data suggest that while iron chelation may play a role in neurite outgrowth, it is not the primary mechanism-of-action; another unidentified functional aspect of ApoTf must also play a role in its neurogenic ability.

The present inventors further sought to determine whether a reduction of transferrin's iron-binding activity by mutation of the N-terminal iron-binding site was sufficient to mediate neurogenesis.

Undifferentiated SH-SYSY cells were treated as described in Example 1. Neurite growth was assessed by imaging and image analysis. At the time of analysis, a 10× solution of Tubulin Tracker (Molecular Probes, T34075) and Hoechst 33342 (Molecular Probes #H3570) nuclear stain was prepared. Briefly, Tubulin Tracker dissolved in DMSO was diluted 1:1 with Pluronic F-127 and further diluted into HBSS to generate a 10× solution. Hoechst 33342 was added to the HBSS-Tubulin Tracker solution at 10 μg/mL to generate at 10× nuclear stain. The 10× staining solution (10 μL) was added directly to treated assay wells and incubated at 37° C. for 30 minutes. Following incubation, 110 μL of 0.4% Trypan Blue was added directly to assay wells and imaged on a Molecular Devices Nano imaging instrument. Nine images/well were acquired in the blue (Nuclei) and green (Tubulin) fluorescent channels for each image. After obtaining images, the MetaExpress Neurite Outgrowth analysis module (Molecular Devices) was used to identify cell bodies and quantify neurites. The total number of neurite branches were divided by the total number of cells imaged to account for different numbers of cells. The Outgrowth Fold Change was determined by setting the untreated control to a value of 1 with all other treatments shown relative to untreated control. All proteins were dosed at a final concentration of 0.2 mg/mL.

Plasma-derived human serum albumin (pdHSA) and ApoTf were obtained and purified from pooled human plasma; recombinant ApoTf (rec ApoTf; SEQ ID NO: 1), and the N-lobe mutant Tf (N-mut rec ApoTf; SEQ ID NO: 4) were obtained by cell culture expression from 293-6E cells.

Briefly, wild-type human transferrin (SEQ ID NO:1) and N-lobe mutant human transferrin (SEQ ID 4) sequences were cloned into mammalian expression plasmids containing N-terminal 6×HIS tag and TEV cleavage sites. The expression plasmids were transfected into the 293-6E cell line, with subsequent harvest of proteins from the cell culture supernatant. Proteins were purified on NI-NTA columns and eluted after washing. TurboTEV protease was used to cleave the N-terminal 6×HIS tag and additional amino acids from the transferrin proteins. Following TEV cleavage, the transferrin proteins were separated from cleaved 6×HIS tag and uncleaved protein by a second Ni-NTA capture column. The flow-through fraction of Ni-NTA capture column was then subject to low pH treatment to remove any potential residual iron bound to these proteins, buffer exchanged to PBS pH 7.4, concentrated, and sterile filtered for final use.

From FIG. 3B we see that plasma-derived human serum albumin (pdHSA) did not affect neurogenesis. However, both ApoTf and recombinant ApoTf did induce neurogenesis of SH-SYSY. The ApoTf mutant (N-mut rec ApoTf) with reduced iron-binding capacity was almost equal to that of ApoTf and rec ApoTf at inducing differentiation of the SH-SYSY cells. Iron-binding does not appear to be the sole mechanism of action for the neurogenic potential of ApoTf.

### Example 4: Neurogenic Effects on SH-SY5Y are Specific to Apo-Transferrin and Apo-Lactoferrin

As the role of iron chelation in ApoTf's neurogenic ability was found to unclear from Example 3 the present inventors determined whether other iron binding proteins can also mediate neurogenesis of SH-SYSY cells.

Undifferentiated SH-SYSY cells were treated as described in Example 1. Neurite growth was assessed by imaging and image analysis. At the time of analysis, a 10× solution of Tubulin Tracker (Molecular Probes, T34075) and Hoechst 33342 dissolved in DMSO was diluted 1:1 with Pluronic F-127 and further diluted into HBSS to generate a 10× solution. Hoechst 33342 was added to the HBSS-Tubulin Tracker solution at 10 μg/mL to generate at 10× nuclear stain. The 10× staining solution (10 μL) was added directly to treated assay wells and incubated at 37° C. for 30 minutes. Following incubation, 110 μL of 0.4% Trypan Blue was added directly to assay wells and imaged on a Molecular Devices Nano imaging instrument. Nine images/well were acquired in the blue (Nuclei) and green (Tubulin) fluorescent channels for each image. After obtaining images, the MetaExpress Neurite Outgrowth analysis module (Molecular Devices) was used to identify cell bodies and quantify neurites. The total number of neurite branches were divided by the total number of cells imaged to account for different numbers of cells. The Outgrowth Fold Change was determined by setting the untreated control to a value of 1 with all other treatments shown relative to untreated control. BSA was obtained from Sigma; rHSA was obtained from Albumedix; ApoTf and HoloTf were obtained and purified from pooled human plasma; Apo-ferritin (equine) was obtained from Sigma; apo-lactoferrin was obtained from Athens Research & Technology. All proteins were dosed at a final concentration of 0.2 mg/mL.

From FIG. 4 we see that neither bovine serum albumin (BSA) nor a low-affinity iron binding form of human serum albumin affected neurogenesis. For further information on the low-affinity iron binding form of human serum albumin (rHSA) see Silva et al., 2009. Biochimica et Biophysica Acta, Vol 1794, p 1449-1458. Holo-transferrin (HoloTf), the iron-saturated form of transferrin, was also unable to induce differentiation of the SH-SYSY cells.

Surprisingly, apo-ferritin, the iron-poor form of ferritin, another high-affinity iron binding protein with multiple iron binding sites, was ineffective at inducing differentiation of the SH-SYSY cells. This furthered the hypothesis that iron binding is the sole mechanism of action for the neurogenic potential of ApoTf. Unexpectedly, apo-lactoferrin also induced differentiation of these cells. Apo-lactoferrin is a structural and functional homologue of apo-transferrin but found in breast milk rather than plasma.

Apo-lactoferrin has 61% identity with apo-transferrin, whereas apo-ferritin and Human Serum Albumin (HSA) are structurally unrelated to either apo-transferrin or apo-lactoferrin.

### Example 5: ApoTf Induced Differentiation of SH-SY5Y Cells is not Through Hypoxia Inducible Factor 1α (HIF-1α)

It has been reported that both ApoTf and HoloTf can induce HIF-1α production leading to associated neuroprotective effects (US2016008437 to Grifols Worldwide Operations limited, the contents of which are incorporated herein by reference). While this is a beneficial attribute prior to death of a neuron, neuroprotection does not benefit the patient once a neuronal cell is dead. Neurogenesis, on the other-hand, benefits the patient after the insult because it can regenerate new neuronal cells.

In substantiation of the premises that ApoTf is mediating neurogenesis outside of the HIF pathway the present inventors tested a well-known, highly specific prolyl hydroxylase (PHD2) inhibitor in the SH-SY5Y cell differentiation assay. 10 λ2 (N-[[1,2-Dihydro-4-hydroxy-2-oxo-1-(phenylmethyl)-3-quinolinyl]carbonyl]-glycine), a small molecule inhibitor of PHD2 is known to activate the HIF pathway through its actions on PHD2. See Chowdhury et al., 2013. ACS Chem. Biol. Vol 8, p 1488. IOX2 has an 1050 of 22 nM for inhibition of PHD2 and can induce up-regulation of HIF-1α in undifferentiated SH-SY5Y with concentrations as little as 1 μM (Ross, US2016008437 supra).

Undifferentiated SH-SY5Y cells were seeded and treated as described in Example 1. Neurite growth was assessed by imaging and image analysis. At the time of analysis, a 10× solution of Tubulin Tracker (Molecular Probes, T34075) and Hoechst 33342 (Molecular Probes #H3570) nuclear stain was prepared. Briefly, Tubulin Tracker dissolved in DMSO was diluted 1:1 with Pluronic F-127 and further diluted into HBSS to generate a 10× solution. Hoechst 33342 was added to the HBSS-Tubulin Tracker solution at 10 μg/mL to generate at 10× nuclear stain. The 10× staining solution (10 μL) was added directly to treated assay wells and incubated at 37° C. for 30 minutes. Following incubation, 110 μL of 0.4% Trypan Blue was added directly to assay wells and imaged on a Molecular Devices Nano imaging instrument. Nine images/well were acquired in the blue (Nuclei) and green (Tubulin) fluorescent channels for each image. After obtaining images, the MetaExpress Neurite Outgrowth analysis module (Molecular Devices) was used to identify cell bodies and quantify neurites. The total number of neurite branches were divided by the total number of cells imaged to account for different numbers of cells. The Outgrowth Fold Change was determined by setting the untreated control to a value of 1 with all other treatments shown relative to untreated control. ApoTf was obtained and purified from pooled human plasma and dosed at a final concentration of 0.2 mg/mL. IOX2 was obtained from Tocris (Cat #4451), resuspended and stored by the manufacturer's recommendations.

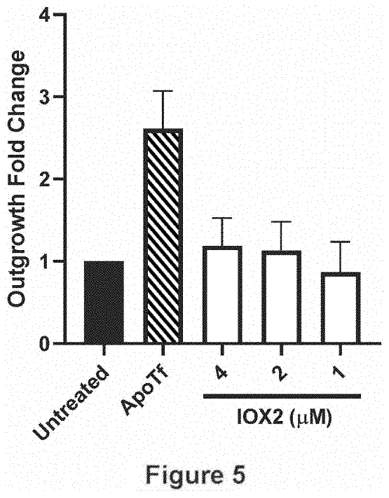

From FIG. 5 it is evident that no neurite outgrowth or differentiation was observed in the IOX2-treated cells. Even at very high concentrations of 4 μM IOX2 no effect was observable (4-fold higher than concentrations reported in US2016008437 to induce of HIF-1α in SH-SY5Y, and over 180-fold higher than the concentration that Chowdhury determined as the IC₅₀ for PHD2 proteins). These data, in combination with the lack of neurogenesis with HoloTf (Example 4), indicate that HIF-1α does not play a role in differentiating SH-SY5Y cells.

### Example 6: Role of Iron Saturation in Transferrin Efficacy

ApoTf, with various purities and iron saturation amounts, as outlined in Table 1 were assessed for their neurogenic potential. The transferrin samples were prepared according to the procedures/methodology known by those skilled in the art and detailed in section 21.4 of L von Bonsdorff, et al., Transferrin, Ch 21, pg 301-310, Production of Plasma Proteins for Therapeutic Use, Eds. J. Bertolini, et al., Wiley, 2013 [Print ISBN:9780470924310 |Online ISBN:9781118356807], the contents of which are incorporated herein by reference.

Protein purity was determined by SDS-PAGE. Iron saturation levels were determined using ICP-AES in accordance with the procedures outlined in Manley et al., J Biol Inorg Chem (2009) 14:61-74, the contents of which are incorporated herein by reference.

TABLE 1

| | Protein | Iron Saturation | |
|---|---|---|---|
| Sample Name | Purity (%) | (%) | Source |
| ApoTransferrin A | 99.11 | 0.27 | Grifols - prepared in |
| | | | house |
| ApoTransferrin B | 98.57 | 0.59 | Grifols - prepared in |
| | | | house |
| ApoTransferrin C | 96.72 | 0.24 | Grifols - prepared in |
| | | | house |
| ApoTransferrin D | 94.35 | Not | Athens Research & |
| | | Determined | Technology Inc., Cat# |
| | | | 16-16A32001-BPG |
| HoloTransferrin | 99.0 | 100 | Grifols - prepared in |
| | | | house |

Undifferentiated SH-SY5Y cells were treated as described in Example 1. Neurite growth was assessed by imaging and image analysis. At the time of analysis, a 10× solution of Tubulin Tracker (Molecular Probes, T34075) and Hoechst 33342 (Molecular Probes #H3570) nuclear stain was prepared. Briefly, Tubulin Tracker dissolved in DMSO was diluted 1:1 with Pluronic F-127 and further diluted into HBSS to generate a 10× solution. Hoechst 33342 was added to the HBSS-Tubulin Tracker solution at 10 μg/mL to generate at 10× nuclear stain. The 10× staining solution (10 μL) was added directly to treated assay wells and incubated at 37° C. for 30 minutes. Following incubation, 110 μL of 0.4% Trypan Blue was added directly to assay wells and imaged on a Molecular Devices Nano imaging instrument. Nine images/well were acquired in the blue (Nuclei) and green (Tubulin) fluorescent channels for each image. After obtaining images, the MetaExpress Neurite Outgrowth analysis module (Molecular Devices) was used to identify cell bodies and quantify neurites. The total number of neurite branches were divided by the total number of cells imaged to account for different numbers of cells. The Outgrowth Fold Change was determined by setting the untreated control to a value of 1 with all other treatments shown relative to untreated control.

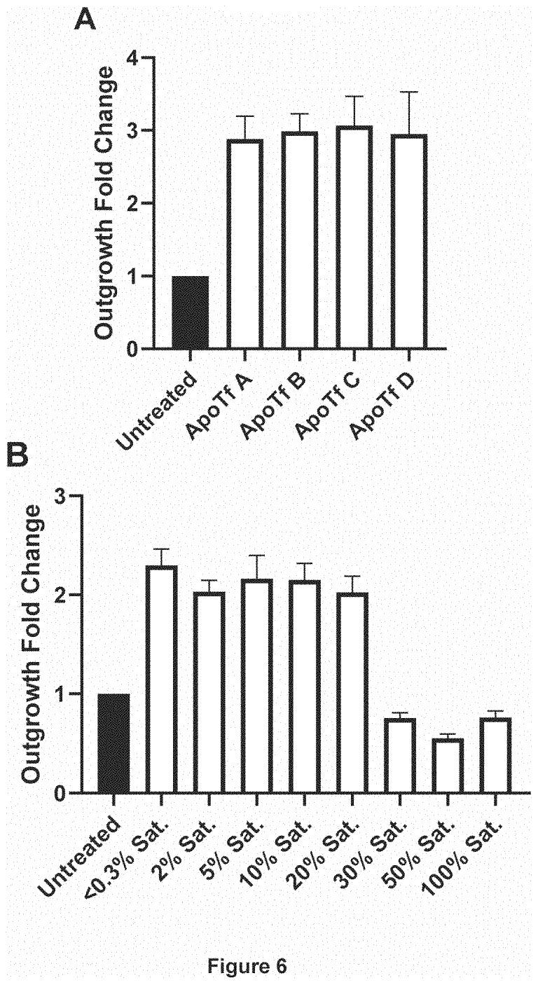

FIG. 6A plots the effect of ApoTf A-D, purity & iron content outlined in Table 1, dosed at a final concentration of 0.2 mg/mL on neurite outgrowth in SH-SY5Y cells. FIG. 6B plots transferrin with various iron saturation levels (listed on the X-axis) dosed at final concentrations of 0.2 mg/mL on neurite outgrowth in SH-SY5Y cells.

ApoTf (<0.3% Saturation) and, HoloTf (100% Saturation) were prepared after purification of transferrin from pooled human plasma as outlined in von Bonsdorff, vide supra. The various iron saturation contents were generated by mixing ApoTf and HoloTf to generate the indicated percent saturations plotted in FIG. 6B.

From FIG. 6A we see that all ApoTf preparations (ApoTf A-D), even the sample with a protein purity of only 94%, were able to induce neurogenic differentiation of SH-SY5Y. FIG. 6B illustrates effect the degree of iron saturation had on the ability of transferrin to induce differentiation of the SH-SY5Y cells. In this example, ApoTf or HoloTf with protein purities of at least 99% were mixed in various ratios to determine the effect of iron saturation/content. Transferrin with an iron saturation content less than 30% showed neurogenic potential.

### Example 7: Apo-Transferrin Acts Synergistically with Neurotrophic Protein and Peptide Factors to Induce Differentiation

Several neurotrophic protein factors have been considered for clinical use for stimulation of neurogenesis in neurodegenerative conditions and after traumatic brain injury. See Houlton et al., 2019. Frontiers in Neurosci., Vol. 13, Article 790; Weissmiller and Wu, 2012. Translational Neurodegeneration, Vol. 1:14; Apfel, 2001. Clin Chem Lab Med., Vol. 39(4), p 351.

Proteins from three neurotrophic superfamilies were tested for function in combination with ApoTf. These neurotrophic proteins are: BDNF (brain-derived neurotrophic factor; NGF superfamily), GNDF (glial cell line-derived neurotrophic factor; TGF-β superfamily), and CNTF (cilliary neurotrophic factor-1; neurokine superfamily). In addition, another known neurotrophic peptide, PACAP (amino acids 1-38 of pituitary adenylate cyclase-activating polypeptide), was assessed for function in combination with ApoTf.

Undifferentiated SH-SY5Y cells were treated as described in Example 1. Neurite growth was assessed by imaging and image analysis. At the time of analysis, a 10× solution of Tubulin Tracker (Molecular Probes, T34075) and Hoechst 33342 dissolved in DMSO was diluted 1:1 with Pluronic F-127 and further diluted into HBSS to generate a 10× solution. Hoechst 33342 was added to the HBSS-Tubulin Tracker solution at 10 μg/mL to generate at 10× nuclear stain. The 10× staining solution (10 μL) was added directly to treated assay wells and incubated at 37° C. for 30 minutes. Following incubation, 110 μL of 0.4% Trypan Blue was added directly to assay wells and imaged on a Molecular Devices Nano imaging instrument. Nine images/well were acquired in the blue (Nuclei) and green (Tubulin) fluorescent channels for each image. After obtaining images, the MetaExpress Neurite Outgrowth analysis module (Molecular Devices) was used to identify cell bodies and quantify neurites. The total number of neurite branches were divided by the total number of cells imaged to account for different numbers of cells. The Outgrowth Fold Change was determined by setting the untreated control to a value of 1 with all other treatments shown relative to untreated control.

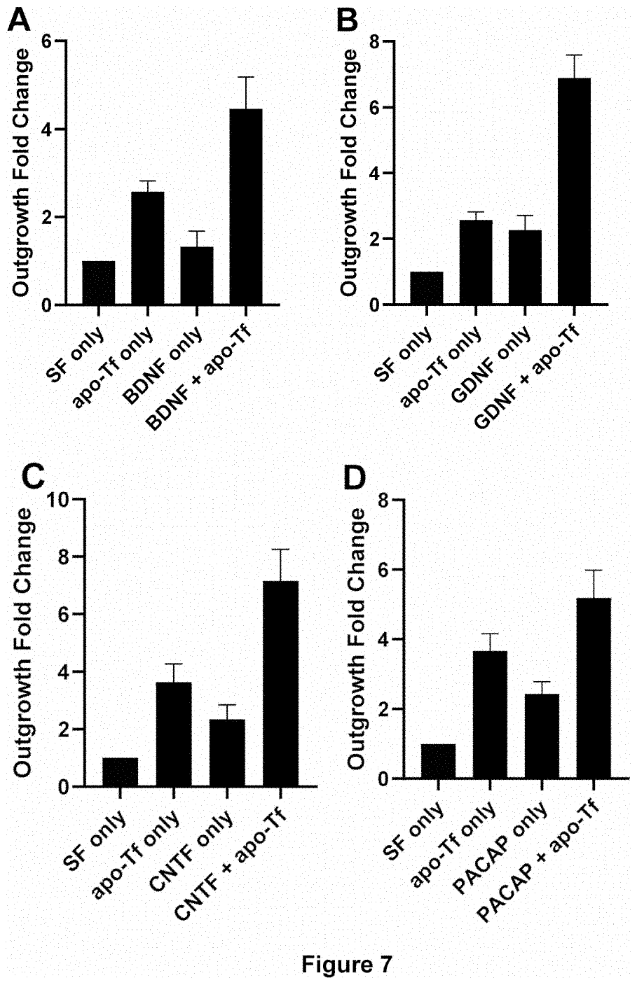

In FIGS. 7A-7D ApoTf was dosed at a final concentration of 0.1 mg/mL either alone or in combination with the indicated neurotrophic factor. (A) BDNF was obtained from Peprotech (Cat #450-02) and dosed at 25 ng/mL. (B) GDNF was obtained from Peprotech (Cat #450-10) and dosed at 1000 ng/mL. (C) CNTF was obtained from Peprotech (Cat #450-13) and dosed at 250 ng/mL. (D) PACAP was obtained from Tocris (Cat #1186) and dosed at 200 nM. The abbreviation SF denotes serum free media.

Reviewing each of FIGS. 7A-7D it is apparent that each of the neurotrophic factors, and the peptide fragment induced differentiation of SH-SYSY cells to different degrees. In some cases, like BDNF, differentiation was not induced by the neurotrophic factor in the absence of ApoTf at the concentrations tested. In the all of the experiments presented, the neurotrophic factors combined with ApoTf induced greater differentiation than the molecules tested alone. Unexpectedly, ApoTf exhibits a synergistic effect with other neurotrophic factors and peptides on neurite outgrowth in SH-SYSY cells.

### Example 8: Apo-Transferrin Acts Synergistically to Induce Differentiation with Neurogenic Small Molecules

The ability of ApoTf to act alongside non-protein based, neurogenic small molecule compounds was tested in Example 7. ApoTf was assessed in combination with the neurogenic compound Y-27632 [trans-4-[(1R)-1-Aminoethyl]-N-4-pyridinylcyclohexanecarboxamide dihydrochloride]. Y-27632 is a Rock1 and Rock2 (Rho kinase) inhibitor. Inhibition of Rock1 and 2 by small molecules has the known ability to induce neuronal differentiation, including SH-SY5Y cells. See Dyberg et al., 2017. PNAS Vol 114 (32), E6603-E6612.

Undifferentiated SH-SY5Y cells were treated as described in Example 1. Neurite growth was assessed by imaging and image analysis. At the time of analysis, a 10× solution of Tubulin Tracker (Molecular Probes, T34075) and Hoechst 33342 (Molecular Probes #H3570) nuclear stain was prepared. Briefly, Tubulin Tracker dissolved in DMSO was diluted 1:1 with Pluronic F-127 and further diluted into HBSS to generate a 10× solution. Hoechst 33342 was added to the HBSS-Tubulin Tracker solution at 10 μg/mL to generate at 10× nuclear stain. The 10× staining solution (10 μL) was added directly to treated assay wells and incubated at 37° C. for 30 minutes. Following incubation, 110 μL of 0.4% Trypan Blue was added directly to assay wells and imaged on a Molecular Devices Nano imaging instrument. Nine images/well were acquired in the blue (Nuclei) and green (Tubulin) fluorescent channels for each image. After obtaining images, the MetaExpress Neurite Outgrowth analysis module (Molecular Devices) was used to identify cell bodies and quantify neurites. The total number of neurite branches were divided by the total number of cells imaged to account for different numbers of cells. The Outgrowth Fold Change was determined by setting the untreated control to a value of 1 with all other treatments shown relative to untreated control. ApoTf was dosed at a final concentration of 0.1 mg/mL either alone or in combination with the indicated small molecule. Y-27632 was obtained from Tocris (Cat #1254) and dosed at 50 μM.

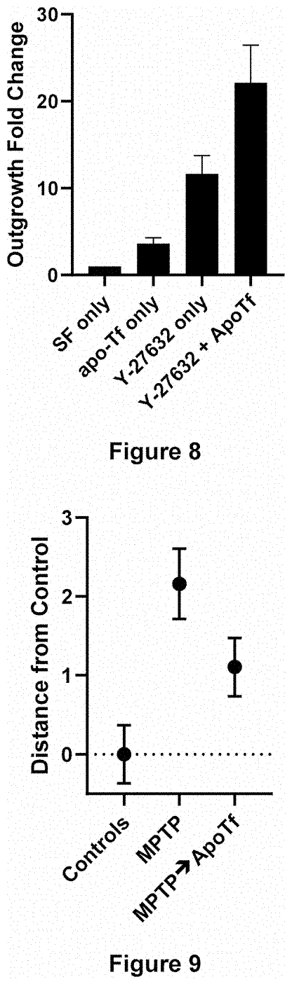

FIG. 8 illustrates that Y-27632 itself is a strongly neurogenic compound, however, in the presence of ApoTf, the neurogenic effect was synergistic showing an effect beyond that exhibited by either molecule alone. The ability of ApoTf to act synergistically with a number of known protein, peptide, and small molecule neurogenic entities is an unexpected and surprising finding.

### Example 9: Improved Gait and Movement by Apo-Transferrin Treatment in a Mouse Model of Parkinson's Disease

To illustrate that the above in-vitro results would successfully translate into positive clinical effects the inventors trialled the therapy in a mouse model of Parkinson's Disease. Mice were administered 1-methyl-4-phenyl-1,2,3,6-tetrahydropyridine (MPTP) to destroy dopaminergic neurons in the substantia nigra and induce Parkinson's disease in the mice. For more detail see Sedelis et al., Behavioural Brain Research 125 (2001), 109-122; Przedborski and Vila, Clinical Neuroscience Research 1 (2001), 407-418.

Destruction of dopaminergic neurons deleteriously effects animal movement. The movement and gait of the mice can be measured by video analysis. As shown in FIG. 9, a substantial alteration in movement and gait was observable in mice exposed to MPTP relative to control mice (n=15). Consistent with the findings in Examples 1-8, MPTP induced Parkinson's Disease in mice was significantly ameliorated by the administration of ApoTf (n=15). FIG. 9 demonstrates the neuroregenerative properties of ApoTf, in that ApoTf greatly improved movement dysfunction in the diseased mice, effectively normalizing the mice to the control animals.

Animal experiments were performed at Charles River Laboratories (Finland), as specified in the license authorized by the national Animal Experiment Board of Finland and according to the National Institutes of Health (Bethesda, Md., USA) guidelines for the care and use of laboratory animals. Eight to twelve-week-old, C57BI/6J mice were housed at a standard temperature (22±1° C.) and in a light-controlled environment (lights on from 7 am to 8 pm) with ad libitum access to food and water.

Solutions of MPTP were prepared by dissolving MPTP hydrochloride in sterile saline at 2.42 mg/mL; corresponding to 2.0 mg/mL of active compound. To induce Parkinson's Disease, the MPTP was given by intraperitoneal injection twice a day at 20 mg/kg. MPTP injections, or saline alone for control mice, were given at 4-hour intervals on two consecutive days (days 0 and 1).

ApoTf protein was administered in sterile PBS, pH 7.4 at a concentration of 51.5 mg/mL. The mice were dosed by intraperitoneal injection with ApoTf at 350 mg/kg or PBS alone for control mice. ApoTf was given once a day on days 1 through 7, with the first ApoTf treatment dose given 1 hour after the last MPTP dose on day 1.

Mice were subjected to kinematic gait analyses on day 7, using a Motorater (TSE Systems GmbH, Bad Homburg, Germany) test system. Animals were tested during their light cycle between 7 am and 8 pm. Before the movement and gait analysis sessions, mice were marked on 31 points of the body to facilitate data analysis of the captured videos. Movement was captured using a high-speed camera (300 fps) from three different directions, from below, and both sides.

The captured videos of each mouse were first converted to the software-readable format. To obtain raw data, the marked points of the body were tracked and each of the three directions were correlated. Thereafter, different gait patterns and movements were extracted using custom-made software developed by Charles River Discovery Research Service Finland. Gait pattern and movement analysis assessed 100 different parameters, including but not limited to stride time, swing time during a stride, speed, step width, stance and interlimb coordination. The data was analysed by using principal component analysis (PCA). The overall gait analysis was based on the PCA of all parameters for each mouse, with the obtained value showing the overall differences, measured as a “distance”, between control mice and MPTP, or MPTP plus ApoTf mice. Control mice (Controls) are set to a value of 0, with the “Distance from Control” shown for MPTP only mice (MPTP), or MPTP mice with subsequent ApoTf treatment (MPTP 4 ApoTf). Values are shown as mean+/−SEM (n=15).

Sequences
The sequences referred to in the preceding
text are outlined below in fasta format.
Human Transferrin [UniProt Q06AH7]
protein sequence
SEQ ID NO: 1
VPDKTVRWCAVSEHEATKCQSFRDHMKSVIPSDGP
SVACVKKASYLDCIRAIAANEADAVTLDAGLVYDA
YLAPNNLKPVVAEFYGSKEDPQTFYYAVAVVKKDS
GFQMNQLRGKKSCHTGLGRSAGWNIPIGLLYCDLP
EPRKPLEKAVANFFSGSCAPCADGTDFPQLCQLCP
GCGCSTLNQYFGYSGAFKCLKDGAGDVAFVKHSTI
FENLANKADRDQYELLCLDNTRKPVDEYKDCHLAQ
VPSHTVVARSMGGKEDLIWELLNQAQEHFGKDKSK
EFQLFSSPHGKDLLFKDSAHGFLKVPPRMDAKMYL
GYEYVTAIRNLREGTCPEAPTDECKPVKWCALSHH
ERLKCDEWSVNSVGKIECVSAETTEDCIAKIMNGE
ADAMSLDGGFVYIAGKCGLVPVLAENYNKSDNCED
TPEAGYFAVAVVKKSASDLTWDNLKGKKSCHTAVG
RTAGWNIPMGLLYNKINHCRFDEFFSEGCAPGSKK
DSSLCKLCMGSGLNLCEPNNKEGYYGYTGAFRCLV
EKGDVAFVKHQTVPQNTGGKNPDPWAKNLNEKDYE
LLCLDGTRKPVEEYANCHLARAPNHAVVTRKDKEA
CVHKILRQQQHLFGSNVTDCSGNFCLFRSETKDLL
FRDDTVCLAKLHDRNTYEKYLGEEYVKAVGNLRKC
STSSLLEACTFRRP
Human Lactoferrin [UniProt P02788]
protein sequence
SEQ ID NO: 2
GRRRRSVQWCTVSQPEATKCFQWQRNMRRVRGPPV
SCIKRDSPIQCIQAIAENRADAVTLDGGFIYEAGL
APYKLRPVAAEVYGTERQPRTHYYAVAVVKKGGSF
QLNELQGLKSCHTGLRRTAGWNVPIGTLRPFLNWT
GPPEPIEAAVARFFSASCVPGADKGQFPNLCRLCA
GTGENKCAFSSQEPYFSYSGAFKCLRDGAGDVAFI
RESTVFEDLSDEAERDEYELLCPDNTRKPVDKFKD
CHLARVPSHAVVARSVNGKEDAIWNLLRQAQEKFG
KDKSPKFQLFGSPSGQKDLLFKDSAIGFSRVPPRI
DSGLYLGSGYFTAIQNLRKSEEEVAARRARVVWCA
VGEQELRKCNQWSGLSEGSVTCSSASTTEDCIALV
LKGEADAMSLDGGYVYTAGKCGLVPVLAENYKSQQ
SSDPDPNCVDRPVEGYLAVAVVRRSDTSLTWNSVK
GKKSCHTAVDRTAGWNIPMGLLFNQTGSCKFDEYF
SQSCAPGSDPRSNLCALCIGDEQGENKCVPNSNER
YYGYTGAFRCLAENAGDVAFVKDVTVLQNTDGNNN
DAWAKDLKLADFALLCLDGKRKPVTEARSCHLAMA
PNHAVVSRMDKVERLKQVLLHQQAKFGRNGSDCPD
KFCLFQSETKNLLFNDNTECLARLHGKTTYEKYLG
PQYVAGITNLKKCSTSPLLEACEFLRK
Y188F Transferrin N-lobe mutant
protein
SEQ ID NO: 3
VPDKTVRWCAVSEHEATKCQSFRDHMKSVIPSDGP
SVACVKKASYLDCIRAIAANEADAVTLDAGLVYDA
YLAPNNLKPVVAEFYGSKEDPQTFYYAVAVVKKDS
GFQMNQLRGKKSCHTGLGRSAGWNIPIGLLYCDLP
EPRKPLEKAVANFFSGSCAPCADGTDFPQLCQLCP
GCGCSTLNQYFGFSGAFKCLKDGAGDVAFVKHSTI
FENLANKADRDQYELLCLDNTRKPVDEYKDCHLAQ
VPSHTVVARSMGGKEDLIWELLNQAQEHFGKDKSK
EFQLFSSPHGKDLLFKDSAHGFLKVPPRMDAKMYL
GYEYVTAIRNLREGTCPEAPTDECKPVKWCALSHH
ERLKCDEWSVNSVGKIECVSAETTEDCIAKIMNGE
ADAMSLDGGFVYIAGKCGLVPVLAENYNKSDNCED
TPEAGYFAVAVVKKSASDLTWDNLKGKKSCHTAVG
RTAGWNIPMGLLYNKINHCRFDEFFSEGCAPGSKK
DSSLCKLCMGSGLNLCEPNNKEGYYGYTGAFRCLV
EKGDVAFVKHQTVPQNTGGKNPDPWAKNLNEKDYE
LLCLDGTRKPVEEYANCHLARAPNHAVVTRKDKEA
CVHKILRQQQHLFGSNVTDCSGNFCLFRSETKDLL
FRDDTVCLAKLHDRNTYEKYLGEEYVKAVGNLRKC
STSSLLEACTFRRP
Y95F/Y188F Transferrin N-lobe
mutant protein
SEQ ID 4
VPDKTVRWCAVSEHEATKCQSFRDHMKSVIPSDGP
SVACVKKASYLDCIRAIAANEADAVTLDAGLVYDA
YLAPNNLKPVVAEFYGSKEDPQTFFYAVAVVKKDS
GFQMNQLRGKKSCHTGLGRSAGWNIPIGLLYCDLP
EPRKPLEKAVANFFSGSCAPCADGTDFPQLCQLCP
GCGCSTLNQYFGFSGAFKCLKDGAGDVAFVKHSTI
FENLANKADRDQYELLCLDNTRKPVDEYKDCHLAQ
VPSHTVVARSMGGKEDLIWELLNQAQEHFGKDKSK
EFQLFSSPHGKDLLFKDSAHGFLKVPPRMDAKMYL
GYEYVTAIRNLREGTCPEAPTDECKPVKWCALSHH
ERLKCDEWSVNSVGKIECVSAETTEDCIAKIMNGE
ADAMSLDGGFVYIAGKCGLVPVLAENYNKSDNCED
TPEAGYFAVAVVKKSASDLTWDNLKGKKSCHTAVG
RTAGWNIPMGLLYNKINHCRFDEFFSEGCAPGSKK
DSSLCKLCMGSGLNLCEPNNKEGYYGYTGAFRCLV
EKGDVAFVKHQTVPQNTGGKNPDPWAKNLNEKDYE
LLCLDGTRKPVEEYANCHLARAPNHAVVTRKDKEA
CVHKILRQQQHLFGSNVTDCSGNFCLFRSETKDLL
FRDDTVCLAKLHDRNTYEKYLGEEYVKAVGNLRKC
STSSLLEACTFRRP
Y426F/Y517F Transferrin C-lobe
mutant protein
SEQ ID NO: 5
VPDKTVRWCAVSEHEATKCQSFRDHMKSVIPSDGP
SVACVKKASYLDCIRAIAANEADAVTLDAGLVYDA
YLAPNNLKPVVAEFYGSKEDPQTFYYAVAVVKKDS
GFQMNQLRGKKSCHTGLGRSAGWNIPIGLLYCDLP
EPRKPLEKAVANFFSGSCAPCADGTDFPQLCQLCP
GCGCSTLNQYFGYSGAFKCLKDGAGDVAFVKHSTI
FENLANKADRDQYELLCLDNTRKPVDEYKDCHLAQ
VPSHTVVARSMGGKEDLIWELLNQAQEHFGKDKSK
EFQLFSSPHGKDLLFKDSAHGFLKVPPRMDAKMYL
GYEYVTAIRNLREGTCPEAPTDECKPVKWCALSHH
ERLKCDEWSVNSVGKIECVSAETTEDCIAKIMNGE
ADAMSLDGGFVYIAGKCGLVPVLAENYNKSDNCED
TPEAGFFAVAVVKKSASDLTWDNLKGKKSCHTAVG
RTAGWNIPMGLLYNKINHCRFDEFFSEGCAPGSKK
DSSLCKLCMGSGLNLCEPNNKEGYYGFTGAFRCLV
EKGDVAFVKHQTVPQNTGGKNPDPWAKNLNEKDYE
LLCLDGTRKPVEEYANCHLARAPNHAVVTRKDKEA
CVHKILRQQQHLFGSNVTDCSGNFCLFRSETKDLL
FRDDTVCLAKLHDRNTYEKYLGEEYVKAVGNLRKC
STSSLLEACTFRRP
BDNF
SEQ ID NO: 6
MFHQVRRVMTILFLTMVISYFGCMKAAPMKEANIR
GQGGLAYPGVRTHGTLESVNGPKAGSRGLTSLADT
FEHVIEELLDEDQKVRPNEENNKDADLYTSRVMLS
SQVPLEPPLLFLLEEYKNYLDAANMSMRVRRHSDP
ARRGELSVCDSISEWVTAADKKTAVDMSGGTVTVL
EKVPVSKGQLKQYFYETKCNPMGYTKEGCRGIDKR
HWNSQCRTTQSYVRALTMDS KKRIGWRFIRIDTS
CVCTLT IKRGR
GDNF
SEQ ID NO: 7
MQSLPNSNGAAAGRDFKMKLWDVVAVCLVLLHTAS
AFPLPAANMPEDYPDQFDDVMDFIQATIKRLKRSP
DKQMAVLPRRERNRQAAAANPENSRGKGRRGQRGK
NRGCVLTAIHLNVTDLGLGYETKEELIFRYCSGSC
DAAETTYDKILKNLSRNRRLVSDKVGQACCRPIAF
DDDLSFLDDNLVYHILRKHSAKRCGCI
CNTF
SEQ ID NO: 8
MAFTEHSPLTPHRRDLCSRSIWLARKIRSDLTALT
ESYVKHQGLNKNINLDSADGMPVASTDQWSELTEA
ERLQENLQAYRTFHVLLARLLEDQQVHFTPTEGDF
HQAIHTLLLQVAAFAYQIEELMILLEYKIPRNEAD
GMPINVGDGGLFEKKLWGLKVLQELSQWTVRSIHD
LRFISSHQTGIPARGSHYIANNKKM
PACAP
SEQ ID NO: 9
HSDGIFTDSYSRYRKQMAVKKYLAAVLGKRYKQRVKNK

1. A method of promoting and or inducing generation of new neural cells in a patient that has suffered a neurodegenerative event,
the method comprising administering a therapeutically effective amount of a protein selected from transferrin, lactoferrin, and combinations thereof to the patient in need thereof.

2. The method of claim 1, wherein the therapeutically effective amount of transferrin or lactoferrin administered to the patient has an iron saturation of less than about 20%.

3. The method of claim 1, wherein the protein is human transferrin.

4. The method of claim 1, wherein the transferrin is plasma-derived or recombinant.

5. The method of claim 4, wherein the recombinant transferrin is a mutant transferrin selected from the group consisting of:
i) Y188F mutant comprising the amino acid sequence set forth in SEQ ID NO: 3;
ii) Y95F/Y188F mutant comprising the amino acid sequence set forth in SEQ ID NO: 4;
iii) Y426F/Y517F mutant comprising the amino acid sequence set forth in SEQ ID NO: 5; and
iv) combinations thereof.

6. The method of claim 1, wherein the transferrin is a domain of a fusion protein, and the fusion partner is an immunoglobulin Fc domain.

7. The method of claim 1, wherein the neurodegenerative event is caused by a neurodegenerative disease.

8. The method of claim 1, wherein the neurodegenerative event is a neurodegenerative disease selected from the group consisting of Parkinson's disease, frontotemporal dementia, Alzheimer's disease, Mild Cognitive Impairment, Diffuse Lewy body disease, Dementia with Lewy bodies type, demyelinating diseases such as multiple sclerosis and acute transverse myelitis, amyotrophic lateral sclerosis, Huntington's disease, Creutzfeldt-Jakob disease, corticobasal ganglionic degeneration, peripheral neuropathy, progressive supranuclear Palsy, spinocerebellar degenerations, spinal ataxia, Friedreich's ataxia, cerebellar cortical degenerations, neurogenic muscular atrophies, anterior horn cell degeneration, infantile spinal muscular atrophy, and juvenile spinal muscular atrophy, subacute sclerosing panencephalitis, Hallervorden-Spatz disease, dementia pugilistica, Pick's disease, tauopathies, synucleinopathies, and combinations thereof.

9-21. (canceled)

22. A method of stimulating neural cell development in a patient that has suffered a neurodegenerative event,
the method comprising administering a therapeutically effective amount of a protein selected from transferrin, lactoferrin, and combinations thereof to the patient in need thereof.

23. The method of claim 22, wherein the therapeutically effective amount of transferrin or lactoferrin administered to the patient has an iron saturation of less than about 20%.

24. The method of claim 22, wherein the protein is human transferrin.

25. The method of claim 22, wherein the transferrin is plasma derived, or recombinant.

26. The method of claim 25, wherein the recombinant transferrin is a mutant transferrin selected from the group consisting of:
i) Y188F mutant comprising the amino acid sequence set forth in SEQ ID NO: 3;
ii) Y95F/Y188F mutant comprising the amino acid sequence set forth in SEQ ID NO: 4;
iii) Y426F/Y517F mutant comprising the amino acid sequence set forth in SEQ ID NO: 5; and
iv) combinations thereof.

27. The method of claim 22, wherein the transferrin is a domain of a fusion protein, and the fusion partner is an immunoglobulin Fc domain.

28. The method of claim 22, wherein the neurodegenerative event is caused by a neurodegenerative disease.

29-42. (canceled)

43. A stable pharmaceutical composition comprising:
a therapeutically effective amount of a protein selected from transferrin, lactoferrin, and combinations thereof, and
at least one pharmaceutically acceptable excipient,
wherein the therapeutically effective amount of a protein selected from transferrin, lactoferrin, and combinations thereof has an iron saturation of less than about 25%.

44. The pharmaceutical composition of claim 43, wherein the protein is human transferrin.

45. The pharmaceutical composition of claim 43, wherein the transferrin is plasma derived, or recombinant.

46. The pharmaceutical composition of claim 45, wherein the recombinant transferrin is a mutant transferrin selected from the group consisting of:
Y188F mutant comprising the amino acid sequence set forth in SEQ ID NO: 3;
Y95F/Y188F mutant comprising the amino acid sequence set forth in SEQ ID NO: 4;
Y426F/Y517F mutant comprising the amino acid sequence set forth in SEQ ID NO: 5; and
combinations thereof.

47. The pharmaceutical composition of claim 43, wherein the transferrin is a domain of a fusion protein, and the fusion partner is an immunoglobulin Fc domain.
